(19) United States
(12) Patent Application Publication
(10) Pub. No.: US 2025/0210166 A1
(43) Pub. Date: Jun. 26, 2025

(54) GENERATION OF CLINICAL MULTIMEDIA REPORTS

(71) Applicant: MEDIPHANY, Shaker Heights, OH (US)

(72) Inventor: Evan Mason, Shaker Heights, OH (US)

(21) Appl. No.: 18/991,940

(22) Filed: Dec. 23, 2024

Related U.S. Application Data
(60) Provisional application No. 63/614,465, filed on Dec. 22, 2023.

(51) Int. Cl. G16H 15/00; G06T 11/60; G06V 30/19; G06V 30/42; G10L 13/027; G10L 15/183; G16H 30/20

(52) U.S. Cl. CPC G16H 15/00; G06T 11/60; G06V 30/19; G06V 30/42; G10L 13/027; G10L 15/183; G16H 30/20; G06T 2210/41; G06V 2201/03

(57) ABSTRACT

Systems and methods are provided for generating clinical multimedia reports. Audio is received from a user describing a set of at least one medical image. At least one visual supplement associated with a subject of the audio received from the user is retrieved from an associated library. A multimedia report describing the medical image or images is generated from the received audio, the set of at least one medical image, and visual supplement or supplements.

### RELATED APPLICATION

This application claims priority to U.S. Provisional Application Ser. No. 63/614,465, filed Dec. 22, 2023. The entire content of this application is incorporated herein by reference in its entirety for all purposes.

### TECHNICAL FIELD

The present invention relates to health information systems, and more particularly, to generation of clinical multimedia reports.

### BACKGROUND

Like any complex field, clinical medical practice has a specific vocabulary that can make discussion of the field difficult for individuals outside of the field, or even outside of a specific specialty within the field. As a result, it can be difficult and time-consuming for physicians and medical technicians to communicate test results, imaging reports, and other clinically relevant information to a patient in a manner easily understood by a non-clinician or even clinicians who are not specialists. This can lead to miscommunications with patients, potentially reducing their satisfaction with medical care.

### SUMMARY

In one example, a method is provided. Audio is received from a user describing a set of at least one medical image. At least one visual supplement associated with a subject of the audio received from the user is retrieved from an associated library. A multimedia report describing at least one medical image is generated from the received audio, the set of at least one medical image, and the at least one visual supplement.

In another example, a system includes a processor, an input device, and a non-transitory computer readable medium storing machine-readable instructions executable by the processor. The machine-executable instructions include a voice transcriber that recognizes words in audio received at the input device describing a set of at least one medical image and records the recognized words as a text transcription on the non-transitory computer readable medium and a library of visual supplements. A report generator generates a multimedia report from the received audio or the transcribed text and one or more visual supplements stored in the library of visual supplements.

In a further example, audio is received from a user describing a set of at least one medical image. A text transcript of the audio received from the user is generated. The text transcript is provided to a machine learning model to generate a layman-oriented text explaining the content of the set of at least one medical image. A multimedia report describing the set of at least one medical image is generated from the layman-oriented text and the set of at least one medical image.

### BRIEF DESCRIPTION OF THE DRAWINGS

FIG. 1 illustrates one example of a system for generating a multimedia clinical report;

FIG. 2 illustrates an example of a system for generating a multimedia report for a patient;

FIG. 3 illustrates an example of a system for generating training materials for a radiologist;

FIG. 4 illustrates one example of a method for generating a multimedia report from one or more medical images;

FIG. 5 illustrates one example of a method for generating a video report for a patient; and

FIG. 6 is a schematic block diagram illustrating an example system of hardware components capable of implementing examples of the systems and methods disclosed herein.

### DETAILED DESCRIPTION

As used herein, a “video” is a group of images arranged in a sequence.

As used herein, a “convolutional neural network” refers to any neural network having at least one convolutional layer.

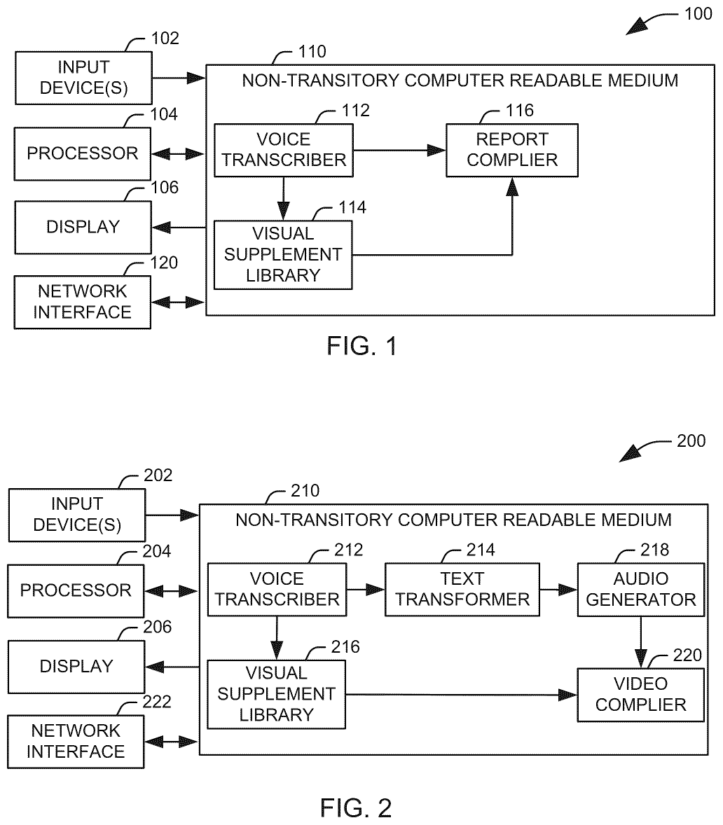

FIG. 1 illustrates one example of a system 100 for generating a multimedia clinical report. The system 100 includes one or more input devices 102, a processor 104, a display 106, and a non-transitory computer readable medium 110. The input device 102 can include any appropriate means for interacting with software executed by the processor, providing audio data to the system 100, or entering text into the system. In one implementation, the input device 102 includes each of a microphone, a keyboard, and a device for interacting with software executed by the processor, such as a mouse, touchpad, or touchscreen. It will be appreciated that the microphone can be used for providing voice commands for interaction with the software.

The non-transitory computer readable medium 110 stores a voice transcriber 112 that recognizes words in received audio and records the recognized words as a text transcription on the non-transitory computer readable medium. It will be appreciated that the voice transcriber 112 can be specifically configured to identify technical terms associated with a given medical field, such as orthopedics, and specifically configurated to identify one or more key terms associated with information stored in a library of visual supplements 114 stored on the non-transitory computer readable medium 110. Each of the visual supplements in the library stored in the library 114 can be a video, image, illustration, three-dimensional model, or animation that is associated with one or more of a structure within the body, a medical procedure, a location of interest within the body, a disorder or illness, or a type of injury. Using the example of orthopedics, a given visual supplement could be a magnetic resonance imaging (MRI) image depicting damage to a meniscus in a left knee. In one example, the visual supplements in the library 114 can be divided into categories (e.g., via a tagging system or folder structure) representing common medical conditions or procedures to allow a clinician to quickly find visual supplements relevant to a given report. It will be appreciated that, in addition to the visual supplements, the images and graphics that are the subject of the report can be, and likely will be, incorporated into the multimedia report.

In some implementations, the system 100 can include a generative model (not shown) that can generate new visual supplements from the transcribed text. In one example, a large language model can be used to summarize the transcribed text, and the resulting summary can be used as an input to the generative model to generate an appropriate visual supplement. For example, a report describing a torn meniscus in the left knee can be reduced to a summary “torn meniscus in the left knee” that is provided as a prompt to the generative model to produce an appropriate explanatory graphic. Any such generated models can be stored in the library 114 for later retrieval and use.

A report compiler 116 generates a multimedia report from the received audio or the transcribed text and one or more visual supplements stored in the library 114. In one implementation, the user can select visual supplements to be added to a video report and associate them with appropriate locations within the received audio or another audio file derived from the received audio. Words and phrases recognized from the received audio at the voice transcriber 112 can be used to automatically select or suggest visual supplements from the library 114 for appropriate points within the audio for the report. Alternatively or additionally, one or more medical images associated with the test can be provided to a machine learning model (not shown) that classifies the image into one of a plurality of classes associated with video supplements, with the selected class used to automatically select or suggest visual supplements from the library 114. In one implementation, a user can pin visual supplements for later use, at which point they are loaded into memory to be immediately available for review and incorporation into the multimedia report.

In one example, visual supplements, text, audio, or video within a given multimedia report can be tagged with labels representing an appropriate audience for the visual supplement, and multiple versions of the reports can be generated based upon the tagged elements. For example, elements can be labeled as “clinician” or “non-clinician,” with a first version of the video including all elements and a second version of the report excluding all “non-clinician” elements. This allows for materials to be included in the report for patient education without burdening a clinician reviewing the report with information with which the clinician is already familiar. In another implementation, elements of the report can be labeled for quality assurance, allowing the user to comment on image quality or imaging protocol and send the report to another medical professional without these comments being visible to clinicians or patients.

In another implementation, a report can be supplemented with additional information that is useful for training another user. This information can be tagged for training and omitted from reports generated for clinicians and patients. The report can be supplemented by visual supplements and other materials, and the report can be assigned tags representing any or all of the imaging modality, the body part, a complexity of the analysis required for the report, and the pathology represented in the image, either by the user or via optical character recognition of the image, analysis of a text transcript of the training report, and analysis of a summary generated of the text transcription via a large language model. The assigned classifications can be used to aggregate the reports into a curriculum or lesson based on groups of reports with similar subjects and complexity and to generate “quizzes” from these groups, for example, based on a prompt from the user. The assigned complexity of each report can be automatically revised according to the performance of trainees on these quizzes, for example, according to the level of training of the users and their performance. In another implementation, an image search, based on thumbnails associated with the reports or imaging cases, can be used to find reports containing similar images or images matching a text prompt, for example, to add a user in a particular difficult interpretation. In one example, the thumbnails are selected by a user. In another example, the thumbnails are automatically generated as the image that is kept on screen for the longest time in the video, either by using optical character recognition to match data across video frames, such as slice numbers and positions within the image, or via direct comparison of the frames to determine which image was present across the most frames.

Further, overlays and other information can be tagged to be capable of toggling for the viewer to allow a medical professional being trained to evaluate images associated with the report before the supplemental information is provided. To support this function, personally identifying information on the report can be tagged to be visible to clinicians and patients, but not to users being trained, and versions of the images stripped of this information can be produced for use in training. Additional tags can be added to the report to allow them to be easily retrieved by imaging modality, location or body part, disease characteristic, or patient characteristics, such as sex, age, or patient history. Interactive questions can be added to the report as well, with the user asked to fill in text fields of the report or select portions of the images associated with a disorder or specific structure.

In another implementation, the library 114 can further store a set of dictation templates, each comprising a plurality of free-text fields, and the report compiler 116 can provide appropriate controls for adding video clips, audio clips, and/or visual supplements to the template. Accordingly, a user can record audio describing the procedure and findings of medical imaging or testing, supplement the audio with visual supplements from the library 114, and insert the resulting video into an appropriate position in the template. In one example, the recorded audio can be transcribed at the voice transcriber 112, and key words within the recorded audio can be used to select a location within the template for one or both of the transcribed text and the audio.

In one implementation, one or more of the free-text fields can be linked to locations within an image being reviewed, such when a location is selected, for example, by hovering a cursor over the region or selecting it with a voice command or input device, any entered text or audio can be associated with the appropriate field. In one implementation, these regions can be generated via an automated segmentation process using a machine learning model (not shown) trained on a set of images segmented and labeled by human experts. Further, additional visual supplements can be generated during the dictation process based upon the selected location. This can be extended to scoring for lesions and tumors as well, by allowing the user to select or highlight a region associated with a tumor or lesion and assigning a grade or score to it by selecting the appropriate score or grade from a menu. It will be appreciated that the lesions or tumors can be segmented automatically or highlighted freehand by the user.

Similarly, a scale of the image can be determined and used to determine one or more size measurements for a segmented or highlighted region representing a lesion. For example, a scale marker within the image can be located, for example, via a template matching algorithm applied with a template associated with a given imaging modality. Any label on the scale can be read using optical character recognition or interpreted according to conventions associated with the appropriate imaging modality. Once the scale is established, structures within the image and annotations made by the user can be automatically measured, with the values for the measurements automatically incorporated into a text report associated with the image.

Additionally or alternatively, the user can simply insert visual supplements from the library 114 into the template, either as clickable buttons or thumbnails or as links within the text of the fields. In some implementations, templates can be automatically populated with one or more fields and/or video clips based on a procedure, test, or body location associated with the template. For example, a template for reporting an MRI of a knee can include an appropriate title field and one or more video clips explaining MRI slices and the structure of the knee. It will be appreciated that, like the visual supplements in the library 114, video clips or visual within a template can be tagged to be selectively visible, such that a report provided to a patient may have different information than a report provided to a clinician. Additionally or alternatively, a text recognition algorithm can be used to locate text within medical images within the report, and a blurring can be applied to each image to obscure patient information within the image. Further, one or more text fields, video clips, or sections of a larger video clip can be selected as the impression for the report. In one implementation, a three-dimensional model of a body part represented by an image can be registered with the image, and any locations pointed to or selected in the image can be mirrored on the three-dimensional model to clarify the location under discussion in the report. Once the report is completed, the user can apply a digital signature and provide the report to a recipient or an electronic health records database (not shown) via a network interface 120.

In one implementation, prior records associated with a given patient can be retrieved via the network interface 120 and processed to allow for comparison studies with a current report. In one example, previous studies for a patient are retrieved and dates and subjects (e.g., location or disorder) of the studies are extracted to determine the relevance of the studies to the current study. Where the studies are stored as database records, the date and subject of the study may be readily available from the appropriate fields.

Where the studies are scans of previous studies, optical character recognition (OCR) can be used to extract metadata, such as a date for the study, a modality of the study, and the subject (e.g., patient and location) of the study, and either OCR or image recognition at a machine learning model (not shown) can be used to determine the subject. A matching process using known variants for various image subjects can be used to ensure that the subject of the study is robust across variations in phrasing, such that a study for a “head” MRI is retrieved in response to a request for “brain” MRIs. Extracted dates can be compared to the patient's birthdate, the current date, and the date of the most recent study, with any matching dates ignored, and any studies having a date before a current study and a same subject can be provided to the user for review. In one implementation, the metadata can be extracted by a large language model that is trained to receive an image, for example, with text representing some or all of the desired metadata and extract the metadata from the image in response to a query.

In one example, a review system can be implemented for quality assurance and quality control. Along with providing an integrated interface for quality control for radiologist readings, the process can be made more efficient by allowing for information to be extracted from the report for use in generating a review of an imaging case, for example, via a video for the radiologist or imaging technician responsible for the case. This can be performed as part of the integration of the review interface into the system, or in one example, optical character recognition or image classification, can be used to extract pertinent data from medical images, such as the name of the patient, the issue shown in the image, the body part shown in the image, and the type of imaging study. It also allows for quicker and asynchronous review of the case for multiple reviews, as comments from reviewers can be input and propagated to other reviewers via the interface to more quickly achieve consensus. Similarly, the radiologist or technician responsible for the case can provide feedback to the reviews, for example, indicating agreement or disagreement with the review or even generating their own video in response. The same interface can also be used to share the report with another radiologist for discussion. In some implementations, a large language model can be used to summarize the video, and the summary can be used to classify a type of the error, such as an error of perception or an error of interpretation.

FIG. 2 illustrates an example of a system 200 for generating a multimedia report for a patient. The system 200 includes one or more input devices 202, a processor 204, a display 206, and a non-transitory computer readable medium 210. The input device 202 can include any appropriate means for interacting with software executed by the processor, providing audio data to the system 200, or entering text into the system. In one implementation, the input device 202 includes each of a microphone, a keyboard, and a device for interacting with specific locations on the display 206, such as a mouse, touchpad, or touchscreen.

The non-transitory computer readable medium 210 stores a voice transcriber 212 that recognizes words in received audio and records the recognized words as a text transcription on the non-transitory computer readable medium. This text transcription can be provided to a text transformer 214 that generates a version of the transcribed text that is suitable for a lay audience. In one implementation, the text transformer 214 is implemented as a language model that is trained to recognize technical terms associated with clinical practice in a given field, such as radiology, and generate a layperson-oriented version of the report that excludes technical jargon and includes additional explanation for medical conditions and procedures. In another example, a more general large language model can be used to simplify the text to a desired level. This layperson-oriented version of the report can be supplemented by inclusion of additional material from a library of visual supplements 216 stored on the non-transitory computer readable medium 210, which can include videos, images, illustrations, three-dimensional models, or animations that are relevant to a structure within the body, a medical procedure, a location of interest within the body, a disorder or illness, or a type of injury that is relevant to the report. In one example, key words from the text transcription can be used to provide a suggested list of visual supplements that are expected to be relevant for a given report. It will be appreciated that some of the content of the report can be dynamically generated by the user or via a generative model using prompts from the user or the transcribed text.

An audio generator 218 can convert the layperson-oriented version of the report into an audio file. In one implementation, the audio generator 218 can use a generative algorithm, trained on samples of a user's voice, that produces audio that mimics the voice of the user. This allows recorded audio and generated audio to be freely combined without an obvious discontinuity between the voices. It will be appreciated, however, that any machine-generated audio can be used. A video compiler 220 generates a report from the generated and/or recorded audio and one or more visual supplements stored in the library 216. It will be appreciated that the visual supplements can be selected by the user, automatically added to the video report at appropriate locations within the audio based on specific words and phrases within the text transcription and/or the layperson-oriented version or suggested to the user based upon words and phrases within the texts. It will be appreciated that the video generated for the original audio may not match the layperson-oriented version of the report. As a result, each audio file can be divided into sentences, using both the natural pauses in speech and semantic analysis of the text transcripts for each audio file, and the lengths of corresponding sentences can be compared. Where discrepancies are found, the video can be sped up or slowed down, for example, by adding or removing video frames with a given image, to synchronize the video with the new, layperson-oriented audio file. The resulting video can be provided to a patient via a network interface 222 or presented to the patient at the display 206.

It will be appreciated that any machine learning models referenced herein can utilize one or more pattern recognition algorithms, each of which analyze one or more of an image, an audio clip, and a vector of input values to generate an output corresponding to the input based on a body of training data provided to the machine learning model. The generated parameter can be stored in a non-transitory computer readable medium, for example, as part of a record in an electronic health records database, or used to suggest a course of action to the user. Where multiple classification or regression models are used, an arbitration element can be utilized to provide a coherent result from the plurality of models.

The training process of a given machine learning model will vary with its implementation, but training discriminative models generally involves a statistical aggregation of training data, comprising an input and a selected output, into one or more parameters that can be used to process the input to provide an output consistent with the training process. For example, a label representing a body part can be paired with an image of the body part to provide a training sample for a model for identifying body parts represented in the image. The training process can be achieved in a federated or non-federated fashion. For rule-based models, such as decision trees, domain knowledge, for example, as provided by one or more human experts or extracted from existing research data, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each of a plurality of output classes. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for a set of output classes for the ANN, with each node having an associated value representing a confidence for one of the associated output classes of the classifier.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

Generative models use a body of samples to learn an underlying pattern or distribution of data to generate new samples consistent with the body of samples. Generative models can be applied, for example, in the context of a large language model for simplifying a dictation for a lay audience, for generating novel samples for training users from a body of available samples, or in voice cloning applications for generating audio at the audio generator.

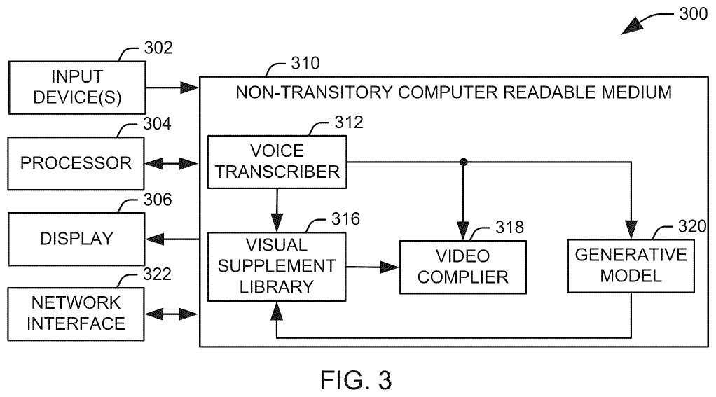

FIG. 3 illustrates an example of a system 300 for generating training materials for a radiologist, such as a teaching file or quality assurance review, in accordance with an aspect of the present invention. The system 300 includes one or more input devices 302, a processor 304, a display 306, and a non-transitory computer readable medium 310. The input device 302 can include any appropriate means for interacting with software executed by the processor, providing audio data to the system 300, or entering text into the system. In one implementation, the input device 302 includes each of a microphone, a keyboard, and a device for interacting with specific locations on the display 306, such as a mouse, touchpad, or touchscreen.

The non-transitory computer readable medium 310 stores a voice transcriber 312 that recognizes words in received audio and records the recognized words as a text transcription on the non-transitory computer readable medium. The recorded audio can be supplemented by inclusion of additional material from a library of visual supplements 316 stored on the non-transitory computer readable medium 310, which can include videos, images, illustrations, three-dimensional models, or animations that are relevant to a structure within the body, a medical procedure, a location of interest within the body, a disorder or illness, or a type of injury that is relevant to the training material. In one example, key words from the text transcription can be used to provide a suggested list of visual supplements that are expected to be relevant for a given report. Further, where the training material is a quality assurance review, the available materials can include the image or images from the report that is the subject of the quality assurance report. It will be appreciated that some of the content of the report can be dynamically generated by the user or via a generative model using prompts from the user or the transcribed text.

A video compiler 318 generates a report from the generated and/or recorded audio and one or more visual supplements stored in the library 316. It will be appreciated that the visual supplements can be selected by the user, automatically added to the video report at appropriate locations within the audio based on specific words and phrases within the text transcription or suggested to the user based upon words and phrases within the texts. Where the training material is a teaching file, the user can also add interactive questions at the end covering the material in the teaching report. These questions can be generated manually or via a generative algorithm 320, such as a large language model, by providing one or more of the recorded audio, a text transcript of the recorded audio, or the medical image that is the subject of the report to the generative algorithm. The resulting presentation can be stored at the non-transitory computer readable medium 310 in a report library 322 for later retrieval.

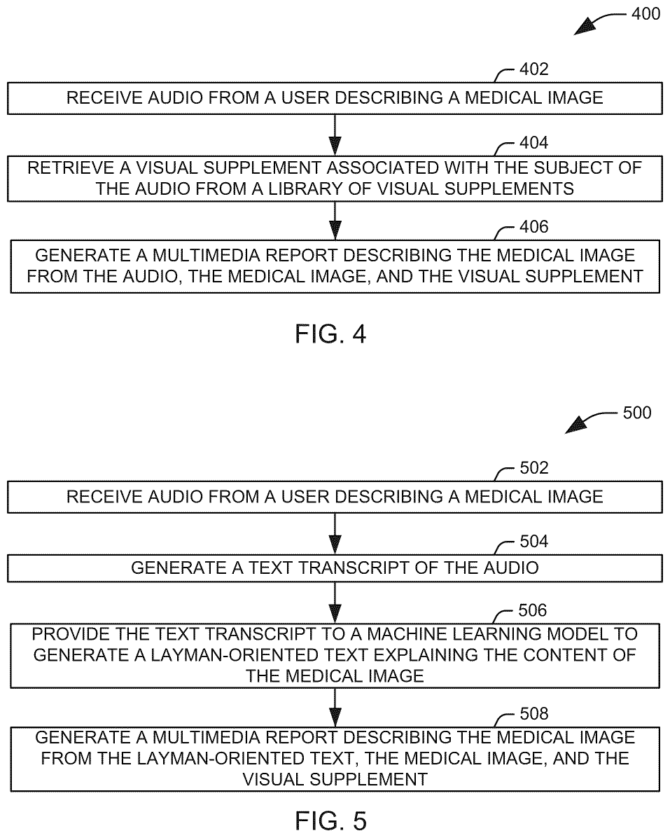

In view of the foregoing structural and functional features described above in FIGS. 1-3, example methods will be better appreciated with reference to FIGS. 4 and 5. While, for purposes of simplicity of explanation, the methods of FIGS. 4 and 5 are shown and described as executing serially, it is to be understood and appreciated that the present invention is not limited by the illustrated order, as some actions could in other examples occur in different orders and/or concurrently from that shown and described herein.

FIG. 4 illustrates one example of a method 400 for generating a multimedia report from one or more medical images. At 402, audio from a user describing a set of at least one medical image is received. At 404, at least one visual supplement associated with a subject of the audio is retrieved from an associated library. In one implementation, a text transcript of the audio is generated, and the visual supplement or supplements associated with the subject of the audio are retrieved according to at least one word in the text transcript. At 406, a multimedia report describing the medical image or images is generated from the received audio, the set of at least one medical image, and the at least one visual supplement. In one example, a text transcript of the audio received from the user is generated and provided to a machine learning model to generate a layman-oriented text explaining the content of the medical image or images. The multimedia report describing the at least one medical image can be generated from the medical image or images, the layman-oriented text, and the visual supplement or supplements.

In one implementation, one or more visual supplements can be labeled by the user such that, when the multimedia report is viewed by a first user, having a first status in a system hosting the multimedia report, the first visual supplement is displayed within the video and when the first visual supplement is viewed by a second user, having a second status in the system hosting the multimedia report, the first visual supplement is not displayed within the video. This can be used primarily to make a video report that is suitable for both patients and clinicians. For example, educational materials that might be useful for a patient, but unnecessary for a clinician, can be labeled to display during video playback only for patients.

In another implementation, the visual supplements can include medical images from past reports for the patient. To this end, metadata can be extracted from a medical image associated with the report, which can include one or more of a date associated with the medical image, a modality of the medical image, and the subject of the medical image. The metadata can be extracted, for example, by applying optical character recognition to the medical image or providing then medical image to a large language model that is trained to receive an image and extract metadata from the received image in response to a query. The extracted metadata can then be used to search a database of radiology reports to find one or more radiology reports related to the given medical image. Further, the metadata extracted from medical images associated with the report can be used to populate fields in a text report associated with the image, such as the patient's name, the date of the scan, and similar information.

FIG. 5 illustrates one example of a method 500 for generating a video report for a patient. At 502, audio from a user describing a set of at least one medical image is received, and at 504, a text transcript of the audio is generated. At 506, the text transcript is provided to a machine learning model to generate a layman-oriented text explaining the content of the set of medical images. In one example, an audio file reciting the layman-oriented text is digitally generated, for example, by using a voice cloning application to recite the layman-oriented text in the user's voice. At 508, a multimedia report describing the set of medical images is generated from the layman-oriented text and the set of at least one medical image. In one example, at least one visual supplement associated with a subject of the audio received from the user audio is retrieved from a library of visual supplements according to at least one word in the text transcript, and the multimedia report can be generated from the layman-oriented text, the set of at least one medical image, and the at least one visual supplement.

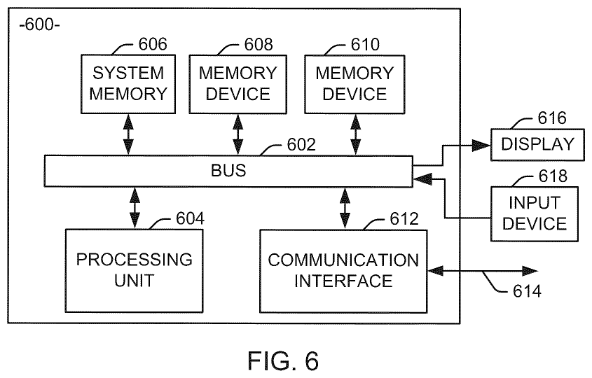

FIG. 6 is a schematic block diagram illustrating an example system 600 of hardware components capable of implementing examples of the systems and methods disclosed herein. For example, the system 600 can be used to implement the systems of FIGS. 1-3. The system 600 can include various systems and subsystems. The system 600 can include one or more of a personal computer, a laptop computer, a mobile computing device, a workstation, a computer system, an appliance, an application-specific integrated circuit (ASIC), a server, a server BladeCenter, a server farm, etc.

The system 600 can include a system bus 602, a processing unit 604, a system memory 606, memory devices 608 and 610, a communication interface 612 (e.g., a network interface), a communication link 614, a display 616 (e.g., a video screen), and an input device 618 (e.g., a keyboard, touch screen, and/or a mouse). The system bus 602 can be in communication with the processing unit 604 and the system memory 606. The additional memory devices 608 and 610, such as a hard disk drive, server, standalone database, or other non-volatile memory, can also be in communication with the system bus 602. The system bus 602 interconnects the processing unit 604, the memory devices 606 and 610, the communication interface 612, the display 616, and the input device 618. In some examples, the system bus 602 also interconnects an additional port (not shown), such as a universal serial bus (USB) port.

The processing unit 604 can be a computing device and can include an application-specific integrated circuit (ASIC). The processing unit 604 executes a set of instructions to implement the operations of examples disclosed herein. The processing unit can include a processing core.

The additional memory devices 606, 608, and 610 can store data, programs, instructions, database queries in text or compiled form, and any other information that may be needed to operate a computer. The memories 606, 608 and 610 can be implemented as computer-readable media (integrated or removable), such as a memory card, disk drive, compact disk (CD), or server accessible over a network. In certain examples, the memories 606, 608 and 610 can comprise text, images, video, and/or audio, portions of which can be available in formats comprehensible to human beings.

Additionally, or alternatively, the system 600 can access an external data source or query source through the communication interface 612, which can communicate with the system bus 602 and the communication link 614.

In operation, the system 600 can be used to implement one or more parts of a system in accordance with the present invention. Computer executable logic for implementing the diagnostic system resides on one or more of the system memory 606, and the memory devices 608 and 610 in accordance with certain examples. The processing unit 604 executes one or more computer executable instructions originating from the system memory 606 and the memory devices 608 and 610. The term “computer readable medium” as used herein refers to a medium that participates in providing instructions to the processing unit 604 for execution. This medium may be distributed across multiple discrete assemblies all operatively connected to a common processor or set of related processors.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

What have been described above are examples of the present invention. It is, of course, not possible to describe every conceivable combination of components or methodologies for purposes of describing the present invention, but one of ordinary skill in the art will recognize that many further combinations and permutations of the present invention are possible. While certain novel features of this invention shown and described below are pointed out in the annexed claims, the invention is not intended to be limited to the details specified, since a person of ordinary skill in the relevant art will understand that various omissions, modifications, substitutions and changes in the forms and details of the invention illustrated and in its operation may be made without departing in any way from the spirit of the present invention. Accordingly, the present invention is intended to embrace all such alterations, modifications, and variations that fall within the scope of the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements. No feature of the invention is critical or essential unless it is expressly stated as being “critical” or “essential.”

What is claimed is:

1. A method comprising:
receiving audio from a user describing a set of at least one medical image;
retrieving, from an associated library, at least one visual supplement associated with a subject of the audio received from the user; and
generating a multimedia report describing the set of at least one medical image from the received audio, the set of at least one medical image, and the at least one visual supplement.

2. The method of claim 1, further comprising generating a text transcript of the audio received from the user, wherein retrieving the at least one visual supplement associated with the subject of the audio received from the user comprises retrieving the at least one visual supplement associated with the subject of the audio according to at least one word in the text transcript.

3. The method of claim 2, wherein retrieving the at least one visual supplement associated with the subject of the audio according to at least one word in the text transcript comprises providing the text transcript to a large language model to generate a summary of the text and providing the summary of the text to a generating model to generate the at least one visual supplement.

4. The method of claim 1, further comprising:
generating a text transcript of the audio received from the user; and
providing the text transcript to a machine learning model to generate a layman-oriented text explaining the content of the set of at least one medical image;
wherein generating the multimedia report describing the at least one medical image comprises generating the multimedia report describing the at least one medical image from the layman-oriented text, the set of at least one medical image, and the at least one visual supplement.

5. The method of claim 4, further comprising digitally generating an audio file reciting the layman-oriented text via a voice cloning application, wherein generating the multimedia report comprises generating the multimedia report from the audio file, the set of at least one medical image, and the at least one visual supplement.

6. The method of claim 1, wherein a first visual supplement of the at least one visual supplement is provided with a label by the user such that, when the multimedia report is viewed by a first user, having a first status in a system hosting the multimedia report, the first visual supplement is displayed within the video and when the first visual supplement is viewed by a second user, having a second status in the system hosting the multimedia report, the first visual supplement is not displayed within the video.

7. The method of claim 1, further comprising extracting metadata from a given medical image of the set of at least one medical image, wherein the metadata includes one or more of a date of associated with the given medical image, a modality of the given medical image, and the subject of the given medical image.

8. The method of claim 7, wherein extracting metadata from the given medical image comprises applying optical character recognition to the given medical image.

9. The method of claim 7, wherein extracting metadata from the given medical image comprises providing the given medical image to a large language model that is trained to receive an image and extract metadata from the received image in response to a query.

10. The method of claim 7, further comprising searching a database of radiology reports using the extracted metadata to find a radiology report related to the given medical image.

11. The method of claim 1, further comprising generating a set of interactive questions at the end of the multimedia report from the received audio.

12. The method of claim 11, wherein generating the set of interactive questions comprises providing one of the received audio, a text transcript of the received audio, and the set of at least one medical image to a generative algorithm.

13. A system comprising:
a processor;
an input device; and
a non-transitory computer readable medium storing machine-readable instructions executable by the processor, the machine-executable instructions comprising:
a voice transcriber that recognizes words in audio received at the input device describing a set of at least one medical image and records the recognized words as a text transcription on the non-transitory computer readable medium;
a library of visual supplements; and
a report generator that generates a multimedia report from the received audio or the transcribed text and one or more visual supplements stored in the library of visual supplements.

14. The system of claim 11, wherein the report generator retrieves the at least one visual supplement associated with the subject of the audio according to at least one word in the text transcript.

15. The system of claim 11, further comprising a machine receiving model that generates a version of the transcribed text that is suitable for a lay audience, the report generator generating the multimedia report from the received audio or the transcribed text and one or more visual supplements stored in the library of visual supplements.

16. The system of claim 1, further comprising a machine learning model that classifies an image of the set of at least one medical image into one of a plurality of classes, the report generator retrieving the at least one visual supplement according to the selected class.

17. A method comprising:
receiving audio from a user describing a set of at least one medical image;
generating a text transcript of the audio received from the user;
providing the text transcript to a machine learning model to generate a layman-oriented text explaining the content of the set of at least one medical image; and
generating a multimedia report describing the at least one medical image from the layman-oriented text and the set of at least one medical image.

18. The method of claim 17, further comprising retrieving, from an associated library, at least one visual supplement associated with a subject of the audio received from the user audio according to at least one word in the text transcript; and
generating a multimedia report describing the at least one medical image from the layman-oriented text, the set of at least one medical image, and the at least one visual supplement.

19. The method of claim 17, further comprising digitally generating an audio file reciting the layman-oriented text.

20. The method of claim 19, wherein digitally generating the audio file reciting the layman-oriented text comprises generating an audio file reciting the layman-oriented text in a voice of the user via a voice cloning application.
